(19) United States
(12) Plant Patent Application Publication
(10) Pub. No.: US 2018/0020601 P1
(43) Pub. Date: Jan. 18, 2018

(54) Begonia plant named 'Fibegdraeven'

(50) ;

(71) Applicant: FIDES B.V., De Lier (NL)

(72) Inventor: Nadine Rijk, De Lier (NL)

(73) Assignee: FIDES B.V., De Lier (NL)

(21) Appl. No.: 14/999,902

(22) Filed: Jul. 14, 2016

(51) Int. Cl. A01H 5/02

(52) U.S. Cl. CPC A01H 5/0238
USPC PLT/349

(57) ABSTRACT

A new and distinct cultivar of Begonia plant named ‘Fibegdraeven’, characterized by its upright to spreading and mounded plant habit; relatively compact; freely basal branching habit; dark green-colored leaves; freely and continuously flowering habit; and double flowers that are light red in color.

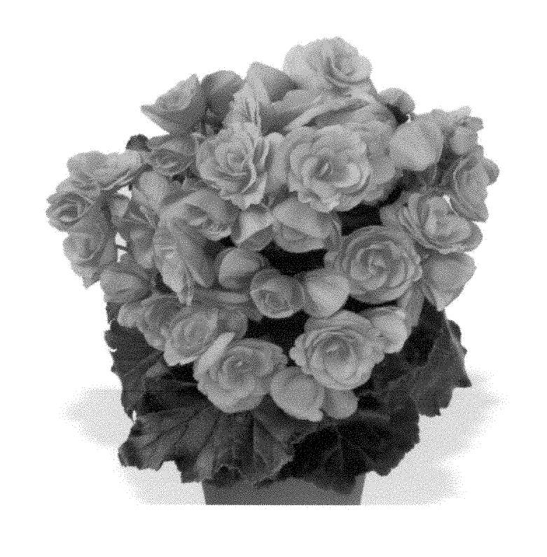

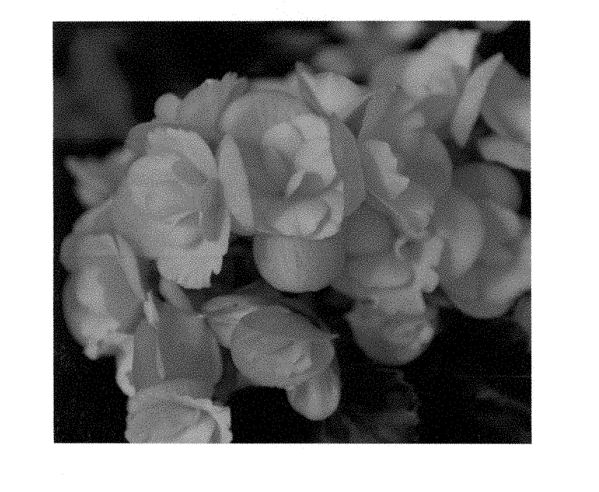

Botanical designation: Begonia hybrida.

Cultivar denomination: ‘FIBEGDRAEVEN’.

### BACKGROUND OF THE INVENTION

The present invention relates to a new and distinct cultivar of Begonia plant, botanically known as Begonia hybrida, and hereinafter referred to by the name ‘Fibegdraeven’.

The new Begonia plant is a product of a planned breeding program conducted by the Inventor in Aalsmeer, The Netherlands. The objective of the breeding program was to develop new freely branching and flowering Begonia plants with unique and attractive flower colors.

The new Begonia plant originated from a cross-pollination made by the Inventor in 2011 of two unidentified proprietary selections of Begonia hybrida, not patented. The new Begonia plant was discovered and selected by the Inventor as a single flowering plant from within the progeny of the stated cross-pollination in a controlled greenhouse environment in Aalsmeer, The Netherlands in 2011.

Asexual reproduction of the new Begonia plant by vegetative tip cuttings in a controlled greenhouse environment in Aalsmeer, The Netherlands since 2011 has shown that the unique features of this new Begonia plant are stable and reproduced true to type in successive generations.

### SUMMARY OF THE INVENTION

Plants of the new Begonia have not been observed under all possible combinations of environmental conditions and cultural practices. The phenotype may vary somewhat with variations in environment such as temperature and light intensity, without, however, any variance in genotype.

The following traits have been repeatedly observed and are determined to be the unique characteristics of ‘Fibegdraeven’. These characteristics in combination distinguish ‘Fibegdraeven’ as a new and distinct Begonia plant:
1. Upright to spreading and mounded plant habit; relatively compact.
2. Freely basal branching habit.
3. Dark green-colored leaves.
4. Freely and continuously flowering habit.
5. Double flowers that are light red in color.

Plants of the new Begonia can be compared to plants of the proprietary parent selections. Plants of the new Begonia differ primarily from plants of the parent selections in uniformity as plants of the new Begonia are more uniform than plants of the parent selections. In addition, plants of the new Begonia are more freely flowering than plants of the parent selections.

Plants of the new Begonia can be compared to plants of Begonia hybrida ‘Dragone Dusty Rose’, not patented. In side-by-side comparisons, plants of the new Begonia differ primarily from plants of ‘Dragone Dusty Rose’ in flower color as plants of ‘Dragone Dusty Rose’ have pink-colored flowers.

### BRIEF DESCRIPTION OF THE PHOTOGRAPHS

The accompanying colored photographs illustrate the overall appearance of the new Begonia plant showing the colors as true as it is reasonably possible to obtain in colored reproductions of this type. Colors in the photographs may differ slightly from the color values cited in the detailed botanical description which accurately describe the colors of the new Begonia plant. The photograph on the first sheet is a side perspective view of a typical flowering plant of ‘Fibegdraeven’ grown in a container. The photograph on the second sheet is a close-up view of a typical flowering plant of ‘Fibegdraeven’.

### DETAILED BOTANICAL DESCRIPTION

Plants used for the aforementioned photographs and following observations and measurements were grown in 12-cm containers during the summer in a glass-covered greenhouse in Rheinberg, Germany. During the production of the plants, day temperatures ranged from 17° C. to 30° C. and night temperatures ranged from 10° C. to 20° C. Plants were eight weeks old when the photograph and the description were taken. In the following description, color references are made to The Royal Horticultural Society Colour Chart, 2007 Edition, except where general terms of ordinary dictionary significance are used.
Botanical classification: Begonia hybrida ‘Fibegdraeven’.
Parentage:
   Female, or seed, parent.—Unidentified selection of Begonia hybrida, not patented.
   Male, or pollen, parent.—Unidentified selection of Begonia hybrida, not patented.
Propagation:
   Type.—By vegetative tip cuttings.
   Time to initiate roots, summer.—About 18 days at temperatures about 22° C. to 30° C.
   Time to initiate roots, winter.—About 21 days at temperatures about 22° C. to 30° C.
   Time to produce a rooted young plant, summer.—About 25 days at temperatures about 22° C. to 30° C.
   Time to produce a rooted young plant, winter.—About 28 days at temperatures about 20° C. to 25° C.
   Root description.—Medium in thickness, fibrous; typically whitish grey in color; actual color of the roots is dependent on substrate composition, water quality, fertilizer type and formulation, substrate temperature and physiological age of roots; plants of the new Begonia have not been observed to form tubers.
   Rooting habit.—Moderately freely branching; medium density.
Plant description:
   Plant form and growth habit.—Upright to spreading and mounded plant habit; relatively compact; pendulous with development; freely basal branching with about five to six primary branches; moderately vigorous to vigorous growth habit.
   Plant height.—About 35 cm.
   Plant width.—About 37 cm.
   Lateral branch description.—Length: About 14 cm to 20 cm. Diameter: About 9 mm. Internode length: About 4.5 mm. Texture: Smooth, glabrous. Color: Close to 152D.
   Leaf description.—Arrangement: Alternate, simple. Length: About 14 cm. Width: About 12 cm. Shape: Ovate to lanceolate. Apex: Acute. Base: Cordate. Margin: Serrate. Texture, upper and lower surfaces: Smooth, glabrous. Venation pattern: Palmate; reticulate. Color: Developing leaves, upper surface: Close to 136A. Developing leaves, lower surface: Close to N77B. Fully expanded leaves, upper surface: Close to 136A; venation, close to 143C. Fully expanded leaves, lower surface: Close to N77C; venation, close to 152D. Petioles: Length: About 7 cm. Diameter: About 6 mm. Texture, upper and lower surfaces: Smooth, glabrous. Color, upper and lower surfaces: Close to 152D.
Flower description:
   Flowering habit.—Double flowers arranged in axillary cymes; freely flowering habit with numerous flowers developing per plant; flowers pendulous and face outwardly to downwardly.
   Fragrance.—None detected.
   Natural flowering season.—Plants in full flower about eight weeks after planting; long flowering period, in the garden plants flower freely and continuously throughout the summer in Northern Europe and can be flowered year-round in greenhouses.
   Flower longevity.—Individual flowers last about four weeks on the plant; flowers persistent.
   Inflorescence height (including peduncle).—About 10 cm.
   Inflorescence diameter.—About 14 cm.
   Female flower buds.—Length: About 1.5 cm. Diameter: About 1.2 cm. Shape: Ovoid. Texture: Smooth, glabrous. Color: Close to 50A.
   Female flowers.—Diameter: About 6.9 cm. Depth (height): About 2.4 cm.
   Female flower tepals and tepaloids.—Quantity per flower and arrangement: Typically about 16 per flower arranged in several whorls. Length: About 2.5 cm. Width: About 2.4 cm. Shape: Ovate. Apex: Acute. Base: Cordate. Margin: Entire. Texture, upper and lower surfaces: Smooth, glabrous. Color: When opening, upper surface: Close to 49A. When opening, lower surface: Close to 49B. Fully opened, upper surface: Close to 50A; color does not fade with development. Fully opened, lower surface: Close to 50B; color does not fade with development.
   Female flower pedicels.—Length: About 1.5 cm. Diameter: About 3 mm. Aspect: About 90° from vertical. Strength: Moderately strong. Texture: Smooth, glabrous. Color: Close to 152D.
   Female flowers reproductive organs.—None observed.
   Fruits and seeds.—Fruit and seed development have not been observed on plants of the new Begonia.
   Male flower buds.—Length: About 1.6 cm. Diameter: About 1.1 cm. Shape: Ovoid. Texture: Smooth, glabrous. Color: Close to 50A.
   Male flowers.—Diameter: About 4.5 cm. Depth (height): About 1.6 cm.
   Male flower tepals and tepaloids.—Quantity per flower and arrangement: Typically about 12 to 14 arranged in several whorls. Length: About 3.3 cm. Width: About 3.5 cm. Shape: Ovate. Apex: Acute. Base: Cordate. Margin: Entire. Texture, upper and lower surfaces: Smooth, glabrous. Color: When opening, upper surface: Close to 49A. When opening, lower surface: Close to 49B. Fully opened, upper surface: Close to 50A; color does not fade with development. Fully opened, lower surface: Close to 50B; color does not fade with development.
   Male flower pedicels.—Length: About 3 cm. Diameter: About 2 mm. Aspect: About 90° from vertical. Strength: Moderately strong. Texture: Smooth, glabrous. Color: Close to 152D.
   Male flowers reproductive organs.—None observed; all transformed into tepaloids.
Disease & pest resistance: Resistance to pathogens and pests common to Begonia plants has not been observed on plants of the new Begonia.
Temperature tolerance: Plants of the new Begonia have been observed to tolerate temperatures ranging from about 4° C. to about 35° C.

It is claimed:

1. A new and distinct Begonia plant named ‘Fibegdraeven’ as illustrated and described.
